(19) United States
(12) Patent Application Publication
(10) Pub. No.: US 2004/0166083 A1
(43) Pub. Date: Aug. 26, 2004

(54) Personal care water-in-oil emulsion products

(75) Inventor: Eric S. Abrutyn, Anderson, OH (US)

Correspondence Address: FROST BROWN TODD LLC, 2200 PNC Center, 201 E. Fifth Street, Cincinnati, OH, 45202-4182, US

(73) Assignee: The Andrew Jergens Company

(21) Appl. No.: 10/375,819

(22) Filed: Feb. 26, 2003

(51) Int. Cl. A61K 7/075; A61K 7/08

(52) U.S. Cl. 424/70.23

(57) ABSTRACT

Personal care products in the form of a water-in-oil emulsion are disclosed. The emulsion utilizes a phosphate ester emulsifier which is optionally combined with a polyoxyalkylene modified siloxane co-emulsifier to form an aesthetically pleasing, storage stable, topically effective personal care composition. The compositions may be formulated to include a wide variety of topically active materials in either the water or oil phase or both, such as deodorant actives, antiperspirant actives, moisturizers, anti-wrinkle actives, sunscreens, hair conditioners, and anti-inflammatory agents.

### TECHNICAL FIELD

The present invention relates to personal care compositions, such as skin care, antiperspirant or hair care compositions, made in the form of a water-in-oil emulsion.

### BACKGROUND OF THE INVENTION

Personal care products comprise a large market both in the United States and worldwide. Such compositions encompass a very wide range of functionalities including, for example, skin care compositions (such as moisturizers, anti-wrinkle compositions and tanning compositions), deodorant and antiperspirant compositions, and hair care compositions (such as conditioning, coloring or styling compositions). Although these compositions all exhibit different functionalities and are applied in somewhat different contexts, they are all formulated, manufactured and sold under a similar set of constraints: they must be safe and effective when applied to the skin or hair; they must present the user with good aesthetics when applied to the skin or hair; they must be storage stable; and they must be cost-effective so that they can be sold for a consumer-acceptable price while allowing the manufacturer to recoup a fair profit.

Formulation of such compositions can be a bit tricky since some of the commonly used ingredients are oil-based or otherwise hydrophobic, while other components are water-based or otherwise hydrophilic. Emulsions, and particularly water-in-oil emulsions, have been utilized frequently for personal care compositions. Such compositions have utilized a wide variety of emulsifiers both alone and in combination. In particular, water-in-silicone emulsions have used dimethicone copolyols (polyoxyalkylene modified siloxanes—either branched or ABA types) to create stable emulsions. These emulsions sometimes need a co-emulsifier to achieve adequate stability. The co-emulsifiers are typically high HLB alkoxylated alcohols. Early work by Dow Corning, later commercialized as Dow 3225C (a blend of PEG/PPG-18/18 dimethicone and cyclomethicone) is one of the earliest water-in-silicone applications for antiperspirant and cosmetic compositions.

Examples of emulsion-based personal care compositions known in the art include the following:

U.S. Pat. No. 6,235,298 B1, Naser et al., issued May 22, 2001, defines a multiple emulsion system that can include skin or hair care agents. The water-in-oil-in-water emulsions utilize a specifically defined external surfactant system together with a stabilizing natural gum polymer. The water-in-oil (inner) emulsion uses a low HLB emulsifier which can be dimethicone copolyol; there is no suggestion to use phosphate esters. The external aqueous phase can include alkyl phosphate esters as one of the hundreds of surfactants disclosed; phosphate esters are not used in any of the examples. Further, there is no suggestion to use alkyl phosphate esters in the inner water-in-oil phase or together with dimethicone copolyols as emulsifiers for any purpose. See also U.S. Pat. No. 6,290,943 B1, Naser et al., issued Sep. 18, 2001.

U.S. Pat. No. 6,268,322 B1, St. Lewis et al., issued Jul. 31, 2001, describes a multiple chambered dispenser which dispenses a surfactant composition and a water-in-oil-in-water multiple emulsion composition. The surfactant composition may include phosphate esters. The multiple emulsion composition may include dimethicone copolyol as the water-in-oil emulsifier and may include phosphate esters in the external phase. There is no suggestion to use dimethicone copolyol together with phosphate esters in the internal water-in-oil phase (or for any other purpose). In fact, there is no suggestion to use phosphate esters at all in the internal water-in-oil phase. See also U.S. Pat. No. 6,306,806 B1, St. Lewis et al., issued Oct. 23, 2001.

U.S. Pat. No. 4,568,480, Thir et al., issued Feb. 4, 1986, describes clear microemulsions which utilize fatty acid esters of alkoxylated phenol derivatives as the microemulsifier component. The patent teaches both water-in-oil and oil-in-water microemulsions. The compositions may include from 1-40% of an alkoxylated phosphate ester as an adjunct component. The alkoxylated phosphate esters are not taught as the primary emulsifier component, nor are they taught for use in combination with dimethicone copolyols as the co-emulsifiers.

U.S. Pat. No. 4,247,424, Kuzel et al., issued Jan. 27, 1981, describes stable liquid laundry detergent compositions which include an ethoxylated alcohol surfactant and an amine oxide surfactant. The compositions are in the form of water-in-oil emulsions, and they contain from 5% to 25% of an emulsifier which may be an alkyl phosphate ester. Phosphate esters are not taught for use as emulsifiers at levels below 5%. Phosphate esters are also taught as being useful as suds suppressants in the compositions. The patent does not teach personal care or pharmaceutical compositions.

U.S. Pat. No. 3,992,332, Zenon, issued Nov. 16, 1976, describes the use of alkyl phosphate esters as static control agents for fabrics.

U.S. Pat. No. 6,165,501, Tirosh et al., issued Dec. 26, 2000, describes certain phospholipid materials (which may include a lower alkyl phosphate ester in the head group) used to make liposomes.

U.S. Pat. No. 6,277,797 B1, Glenn, Jr. et al, issued Aug. 21, 2001, describes cleaning and moisturizing compositions made in the form of an oil-in-water emulsion. The composition uses suspended silica to keep the lipid component dispersed in the composition. The composition includes a lathering surfactant which may be an alkyl phosphate ester (among the many surfactants disclosed) at a level of from about 5% to about 30% of the composition.

U.S. Patent Application 2002/0051801 A1, Abu-Jawdeh et al., published May 2, 2002, describes a composition used for the preparation of a water-in-oil emulsion lubricant. The patent describes the use of conventional lubricant emulsifiers, one of which is a phosphate ester; no specific compounds are disclosed, nor is a pharmaceutical or personal care composition disclosed.

U.S. Patent Application 2002/0009471 A1, Yamasaki et al., published Jan. 24, 2002, describes solid stick-type water-in-oil emulsion compositions which comprise hydrogenated jojoba oil, a polyoxyalkylene-modified siloxane surfactant, and water. The compositions may optionally include anionic surfactants as emulsifiers, and phosphate ester salts are among the list of anionic surfactants disclosed.

U.S. Patent Application 2002/0098221 A1, Taranta et al., published Jul. 25, 2002, describes insecticide compositions in the form of oil-in-water emulsions, comprising an insecticide active, a solvent made up of esters of mono- or dicarboxylic acids, an emulsifier system, a film-forming or thickening agent, and water. Anionic surfactants, including phosphate esters, are among the anionic surfactants which are disclosed as being useful in the emulsifier system.

None of the art discussed above discloses personal care compositions in the form of water-in-oil emulsions which utilize low levels of phosphate esters, either alone or in combination with polyoxyalkylene modified siloxanes, as the emulsifier component. Such compositions are effective at incorporating desired active ingredients and provide exceptionally stable emulsions which exhibit excellent aesthetics when applied to the skin or hair.

### SUMMARY OF THE INVENTION

The present invention relates to a composition, particularly a personal care composition adapted for topical application, in the form of a water-in-oil emulsion which comprises an oil phase; a water phase dispersed in said oil phase; from about 0.1% to about 5% of a phosphate ester emulsifier component; and from about 0% to about 10% (preferably from about 0.05% to about 10%) of a silicon-based surfactant co-emulsifier component, such as alkyl and/or alkoxylated methyl polysiloxane copolyols or siloxanes (polyoxyalkylene modified siloxane copolymer or polyoxyalkylene modified silanol copolymers or polyoxyalkylene modified alkyl siloxane copolymers), or amphoteric or quaternary substituted siloxane copolymers. The phosphate ester emulsifier component has the formula

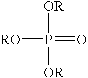

wherein R is selected from H, C₆-C₃₀ straight- or branched-chain alkyl, alkenyl, alkoxy or aralkyl, or C₆-C₁₂ aryl groups, provided that no more than two R groups are hydrogen. In preferred phosphate esters, R is selected from C₁₆-C₂₀ straight- or branched-chain alkyl groups or mixtures thereof (and is most preferably a 50:50 by weight mixture of mono/di-substituted phosphate esters).

As used herein, all percentages and ratios are “by weight” unless otherwise specified.

All patents and publications referred to in this application are incorporated herein by reference, unless otherwise specified.

### DETAILED DESCRIPTION OF THE INVENTION

The present invention relates to compositions, particularly personal care compositions adapted for topical application (for example, to the skin or hair), in the form of a water-in-oil emulsion. These compositions include an oil phase, a water phase dispersed in said oil phase, and from about 0.1% to about 3% of the composition of a phosphate ester emulsifier component. The compositions may optionally contain a silicon-based surfactant co-emulsifier, such as a polyoxyalkylene methyl polysiloxane copolymer. Each of these components will be discussed in detail below.

The Water (Aqueous) Phase

The water phase of the emulsions of the present invention generally make up from about 1% to about 99%, preferably from about 50% to about 82%, of the total emulsion. The water phase generally comprises water, but may additionally optionally include a solute, a topically active compound (which, in this case, would be water-soluble or water-dispersible) and a surfactant. The water phase comprises droplets containing those elements which have a diameter ranging from about 0.01 to about 75 μm and are enveloped by a membrane or film comprising the oil phase.

The aqueous phase may optionally include water-soluble or water-dispersible solutes. Any water-soluble solute (such as polyol- or diol-containing materials) known in the art may be used. Among the solutes which may be added are organic or inorganic salts (such as alkaline metal chlorides, sulfates, nitrates, benzoates and acetates), sugars and sugar derivatives (for example, glucose and sucrose), and polyols (such as glycerin, diglycerin, polyglycerin, propylene glycol, hexylene glycol, pentylene glycol, branched versions of alkylene glycols, polyoxyalkylene modified silicones, diol modified siloxanes, dipropylene glycol, and polypropylene glycol).

It is also possible to add surfactants to the aqueous phase. Typical surfactants, such as anionic, zwitterionic, amphoteric or nonionic surfactants, well known in the art, may be included in the aqueous phase. The aqueous phase can also include optional ingredients traditionally included in topically applied compositions. These optional ingredients include, but are not limited to, dyes, fragrances, preservatives, antioxidants, detackifying agents, and similar types of compounds. These optional ingredients are included in the aqueous phase in an amount sufficient to perform their intended function.

In accordance with an important feature of the present invention, a wide variety of topically active compounds can be incorporated into the water or the oil phase of the emulsion. If found in the water phase, the topically active compounds are water-soluble or water-dispersible and include cosmetic materials and other compounds that act beneficially upon contact with the hair or skin. The topically active compound is present in a sufficient amount to perform its intended function while still maintaining an appropriate margin of safety to the user; typically, the topically active compound is present at from about 0.01% to about 40% by weight, preferably from about 0.05% to about 20% be weight of the aqueous (or the oil) phase.

The topically active compound generally remains on the skin or hair after application, as opposed to being rinsed from the skin or hair. However, particular topically active compounds may be designed to be rinsed from the skin or hair after the compound performs its intended function.

The topically active compound can be incorporated into either the aqueous or the oil phase of the emulsion. Whether a particular topically active compound is incorporated into the aqueous phase or the oil phase is related to the solubility of that topically active compound in water. In preferred embodiments, the topically active compound is water soluble and is incorporated into the aqueous phase. However, a more hydrophobic agent may be used and incorporated into the oil phase.

As used herein, the term “water soluble” means water soluble or water dispersible. A water soluble compound has a water solubility of at least about 0.1 g per ml of water and forms a true solution. A water soluble compound can be inherently water soluble or can be made water soluble by the addition of a solubilizing compound, such as a coupling agent, a co-surfactant, or a solvent. A water dispersible compound remains dispersed in water for at least the time period necessary to manufacture the emulsions of the present invention, i.e., at least about 1 hour.

The topically active compound can be one, or a combination, of a cosmetic compound, a medicinally active compound or any other compound that is useful upon topical application to the skin or hair. Such topically active compounds include, but are not limited to, hair and skin conditioners, hair and skin cleansers, hair fixatives, hair dyes and colorants, hair growth promoters and retardants, deodorants, antiperspirants, skin care compounds, skin moisturizers, skin anti-wrinkle agents, skin self-tanning agents, moisture absorbents, colognes, color cosmetics, permanent wave compounds, hair relaxers, hair straighteners, antibacterial compounds, antifungal compounds, anti-inflammatory compounds, anti-acne compounds, anti-itch compounds, anesthetics, sunscreens, botanical extracts, hydrolyzed vegetable proteins, and other medicinal topically effective compounds. Depending upon the topically active materials incorporated therein, personal care products of the present invention preferably include, for example, topical pharmaceuticals (either prescription or over-the-counter), deodorants, antiperspirants, hair shampoos and conditioners, sunscreens, self-tanning products, and skin care products (for example, cosmetics, moisturizers or anti-wrinkle products).

Examples of topically active materials which may be included in the compositions of the present invention are disclosed in U.S. Pat. No. 6,290,943 B1, Naser et al., issued Sep. 18, 2001, incorporated herein by reference.

The emulsions of the present invention may include an effective amount of an antiperspirant/deodorant active selected from antiperspirant actives, deodorant actives, perfumes, and combinations of those materials. The antiperspirant/deodorant actives are included in the composition in a safe and effective amount, i.e., an amount which is safe to the user but which is sufficient to provide the desired deodorant and/or antiperspirant effects. Antiperspirant actives are generally present at concentrations of from about 0.01% to about 60%, more preferably from about 2% to about 30% by weight of the emulsion. These weight percentages are calculated on an anhydrous metal salt basis exclusive of water and any complexing agents such as glycine, glycine salts, or other complexing agents. The antiperspirant active is formulated in the composition, preferably in the form of a dispersion having a preferred average particle size or diameter of less than about 100 μm, preferably less than about 50 μm. Preferred dispersions have an average particle size or diameter of less than about 5 μm, even more preferably less than about 1.0 μm, and most preferably less than about 0.5 μm. The antiperspirant active for use in the present invention may include any compound, composition or other material having antiperspirant activity. Preferred antiperspirant actives include the astringent metallic salts, especially the inorganic and organic salts, of aluminum, zirconium and zinc, as well as mixtures thereof. Particularly preferred are the aluminum and zirconium salts, such as aluminum halides, aluminum chlorohydrate, aluminum hydroxyhalides, zirconyl oxyhalides, zirconyl hydroxyhalides, and mixtures thereof. Further, these antiperspirant materials can be combined with glycine, urea, propylene glycol, dipropylene glycol, polyethylene glycol, glycerin, etc., as long as they remain effective in reducing sweating in the axillae.

Preferred aluminum salts for use in antiperspirant embodiments of the present invention include those which conform to the formula:

Al₂(OH)ₐCl_{b}.xH₂O

wherein a is about 2 to about 5; the sum of a and b is about 6; x is from about 1 to about 6; and wherein a, b, and x may have non-integer values. Particularly preferred are the aluminum chlorohydroxides referred to as “⅚ basic chlorohydroxide”, wherein a=5, and “⅔ basic chlorohydroxide”, wherein a=4. Processes for preparing aluminum salts are disclosed in U.S. Pat. No. 3,887,692, Gilman, issued Jun. 3, 1975; U.S. Pat. No. 3,904,741, Jones, et al., issued Sep. 9, 1975; U.S. Pat. No. 4,359,456, Gosling, et al., issued Nov. 16, 1982; and British Patent Specification 2,048,229, Fitzgerald, et al., published Dec. 10, 1980, all of which are incorporated herein by reference. Mixtures of aluminum salts are described in British Patent Specification 1,347,950, Shin, et al., published Feb. 27, 1974, which is also incorporated herein by reference.

The antiperspirant embodiments of the present invention contain from about 5% to about 35% (of the emulsion calculated on an anhydrous basis for the antiperspirant active), preferably from about 15% to about 26%, by weight of a particulate antiperspirant material. These weight percentages are calculated on an anhydrous metal salt basis (exclusive of glycine, the salts of glycine, glycols, or other complexing agents). The antiperspirant materials may be impalpable (micronized) or microspherical in form having a particle size ranging about 1 to about 100 microns, more preferably from about 1 to about 45 microns. Any particulate antiperspirant materials known in the art may be used in the present invention. Such materials include, for example, many aluminum or zirconium astringent salts or complexes. Examples of useful antiperspirant materials are described in U.S. Pat. No. 6,287,544, Franklin, et al., issued Sep. 11, 2001; U.S. Pat. No. 6,261,543, Fletcher, et al., issued Jul. 17, 2001; and U.S. Pat. No. 6,187,301, Scavone, et al., issued Feb. 13, 2001, all incorporated herein by reference. Aluminum salts of this type include aluminum chloride and the aluminum hydroxyhalides having the general formula Al₂(OH)ₓQ_{y}.ZH₂O where Q is chlorine, bromine or iodine; where x is from about 2 to about 5, and x+y about 6 (x and y do not need to be integers); and where Z is from about 1 to about 6. Aluminum salts of this type can be prepared in the manner described more fully in U.S. Pat. No. 3,887,692, Gilman, issued Jun. 3, 1975, and U.S. Pat. No. 3,904,741, Jones and Rubino, issued Sep. 9, 1975, incorporated herein by reference. Preferred zirconium salts for use in the antiperspirant embodiments include those which conform to the formula:

ZrO(OH)₂₋ₐClₐ.xH₂O

wherein a is from about 1.1 to about 2.0; x is from about 1 to about 8; and wherein a and x may both have non-integer values.

These zirconium salts are described in Belgian Patent 825,146, Schmitz, issued Aug. 4, 1975, which is incorporated herein by reference. Particularly preferred zirconium salts are those complexes which additionally contain aluminum and glycine, commonly known as “ZAG” or “AZG” complexes. These ZAG complexes contain aluminum chlorhydroxide and zirconyl hydroxychloride (or oxychloride) conforming to the above-described formulas. Such ZAG complexes are described in U.S. Pat. No. 3,679,068, Luedders, et al., issued Feb. 12, 1974; Great Britain Patent Application 2,144,992, Callaghan, et al., published Mar. 20, 1985; and U.S. Pat. No. 4,120,948, Shelton, issued Oct. 17, 1978, all of which are incorporated herein by reference.

The compositions of the present invention may also include deodorant actives, perfumes, or combinations of those materials, at concentrations ranging from about 0.01% to about 60%, preferably from about 0.01% to about 20%, more preferably from about 0.01% to about 10%, and even more preferably from about 0.1% to about 0.5%, by weight of the emulsion. These materials encompass any known or otherwise safe and effective deodorant active or perfume suitable for topical application to human skin.

Deodorant actives suitable for use in deodorant embodiments of the present invention include any topical material that is known for or is otherwise effective in preventing or eliminating malodor associated with perspiration. These deodorant actives are typically antimicrobial agents (e.g., bactericides or fungicides), malodor-absorbing materials, or combinations thereof.

Preferred deodorant actives are antimicrobial agents, non-limiting examples of which include cetyltrimethylammonium bromide, cetyl pyridinium chloride, benzethonium chloride, diisobutylbenzoxyethoxyethyl-dimethylbenzyl ammonium chloride, sodium N-lauryl sarcosine, sodium N-palmethyl sarcosine, lauroyl sarcosine, pentylene glycol (and higher C₆ and C₇ diols), ethylhexyl glycerin, octoxyglycerin, C12-13 alkyl lactate, N-myristoyl glycine, potassium N-lauroyl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (Triclocarban), diaminoalkyl amides (such as L-lysine hexadecyl amide), heavy metal salts of citrate, salicylate, and piroctose (especially zinc salts, and acids thereof), heavy metal salts of pyrithione (especially zinc pyrithione), zinc phenolsulfate, and other zinc salts (PCA, ricinoleate, glycinate), farnesol, phenoxyethanol, and combinations thereof.

Preferred deodorant actives are triclosan, triethylcitrate, pentylene glycol, trimethyl dodecantrianol, and combinations thereof, wherein the preferred concentration of triclosan ranges from about 0.01% to about 5%, more preferably from about 0.1% to about 2%, even more preferably from about 0.1% to about 0.5%, by weight of the emulsion, and wherein the total concentration of deodorant active in a composition ranges from about 0.01% to about 10%, more preferably from about 0.2% to about 2%, even more preferably from about 0.5% to about 1%, by weight of the composition.

Other deodorant actives include odor-absorbing materials such as carbonate and bicarbonate salts, including alkali metal carbonates and bicarbonates, ammonium and tetraalkylammonium, and hydrophilic polyacrylic copolymers. Preferred are sodium and potassium salts of such odor-absorbing materials. Still other deodorant actives include the antiperspirant actives described hereinbefore.

Perfumes suitable for use in the deodorant embodiments of the present invention include any perfume material that can be applied to the skin and is known for or is otherwise effective in masking malodor associated with perspiration, or which otherwise provides the composition with the desired perfumed aroma. These include any perfume or perfume chemical, including pro-perfumes and deo-perfumes, suitable for topical application to the skin. The amount or concentration of the perfume in the deodorant embodiments should be effective to provide the desired aroma characteristics or to mask malodor, wherein the malodor is inherently associated with the composition itself or is associated with malodor development from human perspiration. Perfumes are made by those skilled in the art in a wide variety of fragrances and strengths. Typical perfumes and fragrances are described in Arctander, Perfume and Flavour Chemicals (Aroma Chemicals), Vol. 1 and 11 (1969); and Arctander, Perfume and Flavour Materials of Natural Origin (1960); U.S. Pat. No. 4,322,308, Hooper et al., issued Mar. 30, 1982; U.S. Pat. No. 4,304,679, Hooper et al., issued Dec. 8, 1981; U.S. Pat. No. 5,554,588, Behan et al., issued Sep. 10, 1996; U.S. Pat. No. 4,278,658, Hooper et al., issued Jul. 14, 1981; U.S. Pat. No. 5,501,805, Behan et al., issued Mar. 26, 1996; and EP Patent Application 684 037 A1; all of which are incorporated herein by reference.

### The Oil Phase

The oil phase of the emulsions of the present invention generally make up from about 1% to about 99%, preferably from about 18% to about 50%, by weight of the total emulsion. The oil may be volatile or nonvolatile, and the aqueous phase is dispersed within droplets within the oil phase. Examples of materials which may be included in the oil phase include volatile silicones (both linear or cyclic), volatile hydrocarbons, emollients and high molecular weight nonvolatile silicones, and oil soluble or oil dispersible topically active compounds.

The volatile oil may comprise a volatile hydrocarbon oil or a volatile silicone which evaporates during the process of drying skin or hair, and thereby releases the aqueous phase which can include a topically active compound to contact the skin or hair. A topically active material may also be contained in the oil phase and thereby be released directly when the volatile oil evaporates. Volatile oils include volatile hydrocarbons (such as dodecene, isodecene, hydrogenated polydecene, polydecene, and isohexadecene) and volatile silicone solvents, both of which are well known for use in cosmetic compositions and may be used herein.

The cyclic polydimethylsiloxanes preferably include from about 3 to about 7 silicon atoms, more preferably from about 4 to about 6 silicon atoms. The general formula for such siloxanes is:

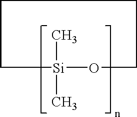

wherein n is from about 3 to about 7. The linear volatile polydimethylsiloxanes contain from about 3 to about 9 silicon atoms and have the general formula: (CH₃)₃—Si—O[Si(CH₃)₂—O]ₙ-Si(CH₃)₃

wherein n is from about 1 to about 6.

Silicones of the above type are commercially available, for example, from Dow Corning Corporation (Dow Corning 344, 345, 200 and 1184 fluids), Union Carbide (Silicone 7207 and Silicone 7158), and Stauffer Chemical (SWS-03314), as well as from General Electric Specialty Chemicals (SF-1202) and Shin Etsu (KF995).

The linear volatile silicone materials generally have viscosities of less than about 5 centistokes at 25° C., while the cyclic materials have viscosities less than about 10 centistokes. “Volatile” means that the material has a measurable vapor pressure. A description of volatile silicones is found in Todd and Byers, “Volatile Silicone Fluids for Cosmetics”, Cosmetics and Toiletries, 91:27-32 (1976), incorporated herein by reference.

Cyclic polydimethylsiloxanes, and particularly cyclomethicone D-5 (decamethylcyclopentasiloxane) and D-6 (dodecamethylcyclohexasiloxane), are preferred for use in the compositions of the present invention.

The oil component, whether volatile or nonvolatile or a mixture of the two, generally comprises from about 1% to about 99%, preferably from about 18% to about 50%, by weight of the emulsion. Volatile hydrocarbons are those which typically contain from about 10 to about 30 carbon atoms and have sufficient volatility to slowly volatilize from the skin or hair after application of the emulsion composition to the skin or hair and subsequent rinsing. A preferred volatile hydrocarbon compound is an aliphatic hydrocarbon having from 12 to about 24 carbon atoms and having a boiling point of about 100° C. to about 250° C. As previously stated, the oil also can be a nonvolatile oil. The nonvolatile oil comprises a nonvolatile silicone compound, a nonvolatile hydrocarbon, or a mixture thereof. Preferably, the nonvolatile oil comprises compounds which contain less than 50% unsaturation. The nonvolatile oil phase does not evaporate from the skin or hair. The topically active compound therefore is released by rubbing the skin or hair to rupture the water-in-oil emulsion. A nonvolatile oil has a boiling point at atmospheric pressure of greater than about 250° C.

Exemplary nonvolatile silicone compounds include polyalkyl siloxanes, polyaryl siloxanes or polyalkylaryl siloxanes. Mixtures of these nonvolatile silicone compounds also are useful. The nonvolatile silicones are nonfunctional siloxanes or siloxane mixtures having a viscosity of about 5 to about 600,000 cs, and typically about 10 to about 10,000 cs, at 25° C. The so-called “rigid silicones,” as described in U.S. Pat. No. 4,902,499, Bolish, Jr. et al., issued Feb. 20, 1990; herein incorporated by reference, having a viscosity above 600,000 cs at 20° C., and a weight average molecular weight of at least about 500,000, also are useful in the compositions of the present invention. Phenyltrimethicone also is useful as a nonvolatile silicone compound. Also useful in the present invention are silicone elastomers commercially available from Dow Corning (9040/9090/9010) and Shin Etsu (KSG 30/42). See U.S. Pat. No. 5,654,362, Schulz, Jr. et al., issued Aug. 5, 1997, incorporated herein by reference. Alkyl-substituted siloxanes may also be used as the nonvolatile silicone component in the compositions of the present invention. See U.S. Pat. No. 5,225,188, Abrutyn, et al., issued Jul. 6, 1993, incorporated herein by reference.

The preferred nonvolatile silicone compound is a nonvolatile polydimethylsiloxane compound, such as a mixture, in about a 2:1 weight ratio, of a low molecular weight polydimethylsiloxane fluid and a higher molecular weight polydimethylsiloxane gum. Preferred silicone gums include linear and branched polydimethylsiloxanes of the following general formula:

(CH₃)₃SiO-[Si(CH₃)₂O]—Si(CH₃)₃,

wherein n is a number from about 2,000 to about 15,000, and preferably from about 2,000 to about 7,000. Silicone gums useful in compositions of the present invention are available from a variety of commercial sources, including General Electric Company, Waterford, N.Y., Dow Corning Corp., Midland, Mich. and Shin Etsu, Japan.

The nonvolatile oil also can comprise a nonvolatile hydrocarbon compound, such as mineral oil. Other exemplary nonvolatile hydrocarbon compounds that can be used as the oil include, but are not limited to, a branched 1-decene oligomer, linear 1-decene dimer or a polydecene.

The oil also optionally can comprise (1) an oil, such as jojoba oil, wheat germ oil or purcellin oil; (2) a water insoluble emollient, such as, for example, an ester having at least about 10 carbon atoms, and preferably about 10 to about 32 carbon atoms; (3) linear or branched-chain C₅-C₅₀ alkyl alcohols, preferably isocetyl or isostearyl alcohol; or (4) dioctyl carbonate or dioctyl ether.

Suitable esters include those comprising an aliphatic alcohol having about three to about thirty carbon atoms and an aliphatic or aromatic carboxylic acid including from two to about twenty carbon atoms, or conversely, an aliphatic alcohol having two to about thirty carbon atoms with an aliphatic or aromatic carboxylic acid including about three to about twenty carbon atoms. The ester is either straight chained or branched. Preferably, the ester has a molecular weight of less than about 500. Suitable esters therefore include, for example, but are not limited to:

(a) aliphatic monohydric alcohol esters, including but not limited to:

myristyl propionate,

isopropyl isostearate,

isopropyl myristate,

isopropyl palmitate,

cetyl acetate,

cetyl propionate,

cetyl stearate,

isodecyl neopentanoate,

cetyl octanoate,

isocetyl stearate;

(b) aliphatic di- and tri-esters of polycarboxylic acids, including but not limited to:

diisopropyl adipate,

diisostearyl fumarate,

dioctyl adipate, and

triisostearyl citrate;

(c) aliphatic polyhydric alcohol esters, including but not limited to:

propylene glycol dipelargonate;

(d) aliphatic esters of aromatic acids, including but not limited to: C₁₂-C₁₅ alcohol esters of benzoic acid, iso-stearyl benzoate,

octyl salicylate,

sucrose benzoate, and

dioctyl phthalate.

Numerous other esters are listed in the International Cosmetic Ingredient and Handbook, 9th edition, 2002, published by CTFA, Washington, D.C., volume 4, incorporated herein by reference.

One of the advantages of the compositions of the present application is that the products can be made to appear clear by matching the refractive index of the oil phase with the refractive index of the water phase (preferably within a±0.001 unit differential between the phases). This can be done, for example, by the addition of an adjuster to one phase of the composition, for example, by the addition of propylene glycol, dipropylene glycol, hexylene glycol or pentylene glycol to the aqueous phase.

Key to the formulation of the compositions of the present invention is the use of a phosphate ester emulsifier component. These phosphate ester emulsifiers are typically used at from about 0.1% to about 5%, preferably from about 0.1% to about 1.5%, more preferably from about 0.3% to about 1.0%, and most preferably about 0.5%, by weight of the emulsion. The phosphate esters useful in the present invention generally have the structural formula:

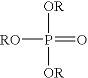

wherein R is selected from H, C₆-C₃₀ straight- or branched-chain alkyl, alkenyl, alkoxy or aralkyl, or C₆-C₁₂ aryl; provided that no more than two R groups per molecule are a hydrogen. The R groups may be substituted or unsubstituted and, for example, may include silicone-containing groups. In preferred phosphate ester emulsifiers used in the present invention, R is C₆-C₃₀ straight- or branched-chain alkyl or alkenyl; more preferably C₆-C₃₀ straight- or branched-chain alkyl; even more preferably C₁₆-C₂₀ straight- or branched-chain alkyl; and most preferably a 50:50 mixture of mono- and di-substituted C₁₆-C₂₀ straight- or branched-chain alkyls. Preferred alkyl phosphate esters are liquids at room temperature. This can be accomplished either through branching (e.g., isocetyl, isostearyl, ethylhexyl, octadecyl, octadodecyl) or unsaturation (e.g., oleyl, linoleyl) in the R group.

The phosphate ester component of the present invention can be the reaction product of a pentavalent phosphorus compound and an alcohol, and the preparation of such materials is well known in the art (see, for example, Crawford et al., U.S. Pat. No. 3,757,864; Poklacki, U.S. Pat. No. 4,007,128, issued Feb. 8, 1977; and Burnham et al., U.S. Pat. No. 4,200,539, issued Apr. 29, 1980; all of which are incorporated herein by reference). The synthesis of these phosphate esters takes place according to a well-known procedure, for example, as set forth in Huddleston, U.S. Pat. No. 5,202,035, issued Apr. 13, 1993, incorporated herein by reference. Alternatively, the phosphate esters useful in the present invention can be prepared by transesterification of orthophosphate esters with triethyl phosphate as shown, for example, in Jones et al., U.S. Pat. No. 5,649,569, issued Jul. 22, 1997, incorporated herein by reference. A dialkyl phosphate can be formed from the reaction product of (1) a polyphosphate intermediate produced by reacting triethyl phosphate and phosphorus pentoxide, and (2) a mixture of aliphatic alcohols having 6 to 10 carbon atoms in their alkyl group. These and other dialkyl phosphates are described in detail throughout U.S. Pat. No. 4,877,894, Huddleston, issued Oct. 31, 1989, which is incorporated herein by reference. The alcohol suitable for reaction with the phosphate intermediates include alkyl alcohols, aralkyl alcohols, ether-containing alkyl alcohols, and aralkyl ether alcohols (or oxyalkylated aralkyl alcohols), and mixtures thereof. It is to be understood that the term “alkyl,” as it applies to the present phosphate esters, includes both straight- and branched-chain alkyl groups. Therefore, when ether alcohols are employed, one or more oxyalkene groups, such as oxyethylene, oxypropylene or oxybutylene is present in the R group of the alcohol designated as ROH. The phosphate ester that is formed in such a reaction is an ether phosphate ester. Thus, the term “phosphate ester” as used herein includes ether phosphate esters. The phosphate ester of the present invention is most preferably non-neutralized. The phosphate ester is most preferably nonaqueous soluble.

Examples of phosphate esters useful in the present invention include, but are not limited to, isostearyl phosphate, isocetyl phosphate, C₉₋₁₅ alkyl phosphate, oleyl phosphate, octadecyl phosphate, isosteareth-2 phosphate, dimethicone PEG-7 phosphate, and vegetable glyceride phosphate. Examples of such materials which are commercially available include Clariant Hostaphat CG120, Croda Crodafos CS2A, Phoenix Pecosil PS-100, and Alzo Dermophos IS-2.

The phosphate esters described above are beneficially used in conjunction with a co-emulsifier selected from alkyl and/or alkoxylated silicon-based surfactants, especially polyoxyalkylene modified siloxanes. These materials can include ester siloxanes (polyoxyalkylene modified siloxane copolymers or polyoxyalkylene modified silanol copolymers or polyoxyalkylene modified alkyl siloxane copolymers), amphoteric or quaternary substituted siloxane copolymers. Combinations of phosphate ester together with such materials provide unexpected benefits in terms of more robust and stable formulations requiring less total emulsifier and simpler processing, as well as wider tolerance between the oil and aqueous phases for refractive index match (clarity). Such co-emulsifiers are included in the emulsions of the present invention at from about 0% to about 10%, preferably from about 0.05% to about 10%, preferably from about 0.5% to about 5%, most preferably from about 0.75% to about 1.5%, by weight of the emulsion. An exemplary oil-soluble silicon-based surfactant is a polyoxyethylene modified siloxane (formerly known as dimethicone copolyol), a compound which is well known in the personal care composition formulation arts. These materials include one or more polyoxyalkylene substitutions on a dimethylsiloxane backbone such that the substitution can occur as an end-cap or along the siloxane chain (or both). These materials also can include polyoxyalkylene modified or polyoxyalkylene and alkyl modified substitutions. The siloxane chain can include from 1 repeating methylsiloxypolyoxyalkylene modified (trisiloxane) to greater than 500 repeating methylsiloxy units where one or more methyls are substituted with polyoxyalkylene or polyoxyalkylene and alkyl groups. Alkyl or alkoxylated methyl polysiloxanes are dimethylsiloxane polymers having polyoxyethylene and/or polyoxypropylene side chains. Examples of such materials include Shin Etsu KF 6017, commercially available from Shin Etsu, Japan and Dow Corning 5225C, commercially available from Dow Corning Company, Midland, Mich. Polyoxyalkylene modified siloxanes conventionally are used in conjunction with silicones because the silicone-containing surfactants are extremely soluble, and a volatile or a nonvolatile silicone compound, are extremely insoluble in water, and have a low skin irritancy potential. Another exemplary, but non-limiting, oil-soluble silicon-based surfactant is an alkyl dimethicone copolyol, such as cetyl dimethicone copolyol, commercially available as ABIL EM 90 from Goldschmidt Chemical corporation, Hopewell, Va. Other examples include Dow Corning 5200 (lauryl PEG/PPG+18/18 methicone), Shin Etsu KSF52 (PEG 10/lauryl dimethicone crosspolymer), and Dow Corning 9011 (PEG 12 dimethicone crosspolymer). Examples of some useful siloxane materials are disclosed in U.S. Application 2002/0009471, Yamasaki et al., published Jan. 24, 2002, incorporated herein by reference.

The alkyl or alkoxylated methyl polysiloxane copolyols, for example, have the structure

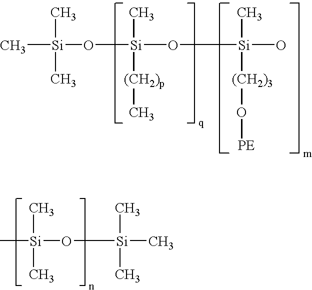

The compositions of the present invention are typically prepared as follows:

1. Add together all oil soluble components (e.g., alkoxylated methyl polysiloxane, phosphate ester, emollients, volatile and non-volatile silicones). Mix at room temperature until uniform (“A” components).

2. In a separate tank mix all aqueous soluble components at room temperature until uniform (“B” components).

3. Add aqueous phase to oil phase with good agitation.

4. Mix at high turbulence for twenty minutes past the completed addition of the aqueous phase. Add fragrance and preservative components (“C” components).

The following non-limiting examples are meant to illustrate the compositions of the present invention, but are not intended to be limiting thereof.

### EXAMPLES

Compositions of the present invention, in liquid form, having the components set forth in the following table, are made using the following procedure:

TABLE 1

| | | % Range | | | | |
|---|---|---|---|---|---|---|
| Ingredient | (Examples) | 1 | 2 | 3 | 4 | Function |
| Cyclomethicone | Dow Corning 245, GE | 9.0 | 7.7 | 8.5 | 2.5 | Transient |
| | SF 1202 | | | | | emollient |
| High molecular | DCC 1502, Shin Etsu | 2.0 | MK 15H | DC9040 | SF1276 | Thickening |
| weight | MK 15H, GE SF | | 2.5 | 2.0 | 2.0 | agent & |
| polydimethylsiloxane | 1276, Shin Etsu SKG | | | | | slip agent |
| (gum or elastomer) | 21, DC 9040 | | | | | |
| Hydrocarbon | hydrogenated | 3.0 | S364 | S364 | P 99A | Emollient |
| | polydecene (Silkflo | | 3.0 | 3.0 | 3.0 | |
| | 362, 364, 366), | | | | | |
| | isododecane | | | | | |
| | (permethyl 101A, | | | | | |
| | isoeicosane (permethyl | | | | | |
| | 102A), polyisobutene | | | | | |
| | (Permethyl 104A), | | | | | |
| | mineral oil, ISOPAR | | | | | |
| | M (C₁₃-C₁₄ | | | | | |
| | isoparaffin) | | | | | |
| Emollient | benzoate esters | 1.5 | F-SB | IsosIson | S56 | Emollient |
| | (Finsolve TN, | | 2.0 | 2.0 | 2.0 | & non- |
| | Finsolve SB, PG-22); | | | | | whitening |
| | fatty esters (isostearyl | | | | | agent |
| | isononanoate, | | | | | |
| | isopropyl PPG-2, | | | | | |
| | isodeceth-7 | | | | | |
| | carboxylate); alkyl | | | | | |
| | methyl siloxanes (C₂₀-C₂₄ | | | | | |
| | AMS, C₁₆ AMS, | | | | | |
| | C₈ AMS); isocetyl | | | | | |
| | alcohol; phenyl | | | | | |
| | functional siloxane | | | | | |
| | (phenyl trimethicone- | | | | | |
| | Shin Etsu 56), isocetyl | | | | | |
| | alcohol, isostearyl | | | | | |
| | alcohol | | | | | |
| Alkyl Phosphate | isostearyl phosphate, | 0.5 | CG-120 | CG-120 | CG-120 | Emulsifier |
| | isocetyl phosphate, C₉-C₁₅ | | 0.5 | 0.5 | 0.5 | |
| | alkyl phosphate, | | | | | |
| | oleyl phosphate, | | | | | |
| | octadecyl phosphate, | | | | | |
| | isosteareth-w | | | | | |
| | phosphate, | | | | | |
| | dimethicone PEG-7 | | | | | |
| | phosphate, vegetable | | | | | |
| | glycerides phosphate | | | | | |
| | (Clariant CG120, Kao | | | | | |
| | SP80I, Pecosil PS- | | | | | |
| | 100, Dermophos IS-2, | | | | | |
| | Arlatone Map 950, | | | | | |
| | Cegosoft VP) | | | | | |
| Emulsifier | PEG-10 dimethicone | 2.0 | KF | EM-97 | DC5525C | Emulsifier |
| | (Shin Etsu KF 6017), | | 6017 | 2.0 | 1.5 | |
| | PPG-18/18 | | 2.0 | | | |
| | dimethicone (DC 3225 | | | | | |
| | or 5225C), bis- | | | | | |
| | PEG/PPG-14/14 | | | | | |
| | dimethicone (Abil | | | | | |
| | EM-97, bix- | | | | | |
| | PEG/PPG-16/16 | | | | | |
| | dimethicone (Abilcare | | | | | |
| | 85), PEG/PPG-20/15 | | | | | |
| | dimethicone (SF1528) | | | | | |
| Co-emulsifier | PEG-12 dimethicone | 0 | 0 | 0 | DC9040 | Emulsifier |
| | crosspolymer (DC | | | | 1.0 | |
| | 9010), PEG-11 methyl | | | | | |
| | ether dimethicone (KF | | | | | |
| | 6011), bix-PEG/PPG- | | | | | |
| | 20/20 dimethicone | | | | | |
| | (Abil 8832, PEG-10), | | | | | |
| | dimethicone/vinyl | | | | | |
| | dimethicone | | | | | |
| | crosspolymer | | | | | |
| | (KGS20), | | | | | |
| | dimethicone/vinyl | | | | | |
| | dimethicone | | | | | |
| | crosspolymer | | | | | |
| | (KSF15), dimethicone | | | | | |
| | crosspolymer | | | | | |
| | (DC9040) | | | | | |
| Water | | q.s. | q.s. | q.s. | q.s. | |
| Aq. Refractive Index | propylene glycol, | 19 | 19 | 19 | 19 | |
| Adjuster | dipropylene glycol, | | | | | |
| | pentylene glycol | | | | | |
| TOTAL | | 100.0 | 100.0 | 100.0 | 100.0 | |

Topically active materials, as described in this application, such as deodorant materials, anti-acne actives, antiperspirant actives, anti-inflammatory materials, skin anti-wrinkling materials, skin moisturizers, color cosmetic materials, hair conditioners, or nutrients for hair or skin, may be incorporated into the water phase or the oil phase of the above compositions as appropriate.

The following example illustrates the formulation of liquid antiperspirant compositions of the present invention. The components listed in the following table are formulated as described above.

TABLE 2

Examples
(wt. %)

| | Ingredient | 5 | 6 |
|---|---|---|---|
| A | Cyclomethicone | 8.75 | 11.5 |
| A | Dimethicone (Shin Etsu MK 15H) | 1.5 | 2.5 |
| A | C₁₂-C₁₅ alkyl benzoate | 1.5 | 1.5 |
| A | Hydrogenated polydecene | 3.0 | — |
| A | PEG-10 dimethicone | 1.25 | 2.0 |
| A | Isostearyl phosphate | 0.5 | 0.5 |
| B | Propylene glycol | 19.0 | 19.0 |
| B | D.I. Water | 14.3 | 10.3 |
| B | AZG solution (43% active concentration) | 50.0 | 52.5 |
| C | Fragrance | 0.2 | 0.2 |

The above compositions, when applied topically to the axillary areas of the user, provides effective antiperspirant performance, good skin feel, no skin or clothing whitening, and good aesthetics to the user, and is storage stable.

The following example illustrates the formulation of a liquid sunscreen composition of the present invention. The components listed in the following table are formulated as described above.

TABLE 3

| | | Example 7 |
|---|---|---|
| Ingredient | | (wt. %) |
| A | Cyclomethicone + PEG/PPG-18/18 dimethicone | 7.5 |
| A | Hydrogenated polydecene | 3.5 |
| A | Octadecyl phosphate | 0.5 |
| A | Isostearyl benzoate | 1.0 |
| A | Decaprylyl maleate | 1.8 |
| A | C₂₄-C₂₈ alkyl methicone | 1.0 |
| A | Octylmethyl cinnamate | 4.0 |
| B | Water | 74.0 |
| B | Acetamide MEA | 2.0 |
| B | Glycerin | 2.0 |
| B | Zinc oxide + cyclomethicone + PEG/PPG-18/18 | 2.0 |
| | dimethicone | |
| C | Preservative | 0.5 |
| C | Fragrance | 0.2 |

The above composition, when applied topically, provides effective sunscreen performance, good skin feel and esthetics to the user and is storage stable.

What is claimed is:

1. A composition in the form of a water-in-oil emulsion which comprises an oil phase; a water phase dispersed in said oil phase; from about 0.1% to about 5% of a phosphate ester emulsifier component; and from 0% to about 10% of a silicon-based surfactant co-emulsifier component.

2. A personal care composition, adapted for topical application, in the form of a water-in-oil emulsion which comprises an oil phase; a water phase dispersed in said oil phase; from about 0.1% to about 5% of a phosphate ester emulsifier component; and from 0% to about 10% of a silicon-based surfactant co-emulsifier component.

3. The personal care composition according to claim 2 wherein the silicon-based surfactant co-emulsifier is present from about 0.05% to about 10% of the emulsion.

4. The personal care composition according to claim 3 wherein the phosphate ester has the formula 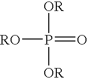
wherein R is selected from H, C₆-C₃₀ straight- or branched-chain alkyl, alkenyl, alkoxy, or alkaryl, or C₆-C₁₂ aryl; provided that no more than two R groups are H.

5. The personal care composition according to claim 4 wherein the phosphate ester is liquid at room temperature.

6. The personal care composition according to claim 5 wherein R is selected from C₆-C₃₀ straight- or branched-chain alkyl or alkenyl.

7. The personal care composition according to claim 6 wherein R is selected from C₆-C₃₀ straight- or branched-chain alkyl.

8. The personal care composition according to claim 7 wherein R is selected C₁₆-C₂₀ straight- or branched-chain alkyl.

9. The personal care composition according to claim 7 wherein the phosphate ester is mixture of mono- and di-substituted esters.

10. The personal care composition according to claim 6 wherein the phosphate ester is present at from about 0.1% to about 1.5% of the emulsion.

11. The personal care composition according to claim 6 wherein the emulsion comprises from about 1% to about 99% of the water phase and from about 1% to about 99% of the oil phase.

12. The personal care composition according to claim 11 wherein the water phase includes a safe and effective amount of water-soluble or water-dispersible topically active material selected from skin care materials, hair care materials, cosmetics, pharmaceuticals, and mixtures thereof.

13. The personal care composition according to claim 11 wherein the oil phase includes a safe and effective amount of an oil-soluble or oil-dispersible topically active material selected from skin care materials, hair care materials, cosmetics, pharmaceuticals, and mixtures thereof.

14. The personal care composition according to claim 11 selected from deodorant compositions, antiperspirant compositions, skin moisturizer compositions, skin anti-wrinkle compositions, sunscreen compositions, hair treatment compositions, hair setting anti-inflammatory compositions, and mixtures thereof.

15. The personal care composition according to claim 11 wherein the refractive indices of the oil phase and the water phase are matched so as to form a clear emulsion.

16. The personal care composition according to claim 11 wherein the oil phase includes volatile material selected from volatile silicones, volatile hydrocarbons, and mixtures thereof.

17. The personal care composition according to claim 16 wherein the volatile material is selected from D3-D7 cyclomethicones, C₁₀-C₃₀ hydrocarbons, and mixtures thereof.

18. The personal care composition according to claim 17 wherein the silicon-based surfactant co-emulsifier is selected from polyoxyalkylene modified siloxanes.

19. The personal care composition according to claim 18 wherein in the polyoxyalkylene modified siloxane is selected from PEG-10 dimethicone, PEG-12 dimethicone crosspolymer, and mixtures thereof.

20. The personal care composition according to claim 7 which comprises from about 0.1% to about 1.5% of the phosphate ester emulsifiers; from about 1.5% to about 3% of the silicon-based surfactant co-emulsifier in the form of a polyoxyalkylene modified siloxane; which includes a topically active material in its water phase, its oil phase, or both phases; and wherein the oil phase includes a volatile material selected from D3-D7 cyclomethicones, C₁₀-C₃₀ hydrocarbons, and mixtures thereof.

21. The personal care composition according to claim 20 wherein the topically active material is selected from deodorant actives, antiperspirant actives, skin moisturizers, skin anti-wrinkle actives, sunscreens, hair conditioners, anti-inflammatory actives, and mixtures thereof.

22. The personal care composition according to claim 21 wherein the refractive indices of the oil phase and water phase are matched to form a clear emulsion.
